(19) United States
(12) Patent Application Publication
(10) Pub. No.: US 2025/0275680 A1
(43) Pub. Date: Sep. 4, 2025

(54) ULTRASOUND-BASED WIRELESS BATTERY-FREE IMPLANTABLE SENSORS COMPRISING A SOLUTION-GATED FIELD-EFFECT TRANSISTOR

(71) Applicants: INSTITUT NATIONAL DE LA SANTE ET DE LA RECHERCHE MEDICALE, PARIS (FR); UNIVERSITE GRENOBLE ALPES, SAINT MARTIN D’HERES (FR); Commissariat à l’énergie atomique et aux énergies alternatives, PARIS (FR)

(72) Inventors: Bruno FAIN, GRENOBLE (FR); Sahil SHARMA, SAINT MARTIN D’HERES (FR); Carole-Anne LERNOUD, SAINT MARTIN D’HERES (FR); Clément HEBERT, SAINT MARTIN D’HERES (FR)

(21) Appl. No.: 18/858,841

(22) PCT Filed: Apr. 28, 2023
(86) PCT No.: PCT/EP2023/061218

§ 371 Date: Oct. 22, 2024

(30) Foreign Application Priority Data
Apr. 28, 2022 (EP) 22305631.8

(51) Int. Cl. A61B 5/00

(52) U.S. Cl. CPC A61B 5/0031

(57) ABSTRACT

Ultrasound-based wireless battery-free implantable sensors comprising a solution-gated field-effect transistor This disclosure relates to wireless battery-free implantable sensors for biomedical applications. Ultrasound-based wireless communication is proposed as an alternative to electromagnetic communication. The implantable sensor comprises a piezoelectric element and a variable impedance load whose value is a function of a physiological parameter of interest. The load is a solution-gated field-effect transistor (SG-FET), preferably a transistor with a graphene channel. The load is connected to the piezo-electric element for wireless communication with an external device. Experiments show that an ultrasound echo signal can be modulated by the gate voltage of the transistor and that the modulated signal can be transmitted through a propagation medium such a biological tissue.

### TECHNICAL FIELD

The present invention relates to wireless and battery free sensors for biomedical applications.

### BACKGROUND

The development of wireless and battery free sensors for biomedical applications is a fast growing research topic and a promising industrial field. Wireless and battery free biosensors are also of great interest for many healthcare monitoring applications and have the potential to improve patient comfort during the diagnosis and the treatment of chronic diseases, among other benefits.

Fully wireless and battery-free implantable sensor technology represents a new step towards the improvement of the patient comfort, especially with implantable sensors, which facilitate insertion surgery and avoid the cumbersome use of wires, large antennas and batteries.

An illustrating example of this progress can be seen in the experiments required to monitor physiological parameters during sleep. Wire-based solutions are often felt as a burden because they reduce patient mobility and degrade the patient's sleep state, which can potentially lead to an inaccurate diagnosis. On the other hand, wireless solutions can be worn without discomfort by the patient, and ultimately provide better and more accurate measurements and diagnosis.

Typically, wireless implantable biosensors comprise a sensing element capable of measuring changes of a physiological parameter of interest, and a wireless communication device, such as a radiofrequency (RF) antenna, capable of transmitting data towards an external communication device placed outside of the patient's body.

So far, most of the wireless biosensor wearable or implantable technologies rely on electromagnetic (EM) wireless communication. EM-based wireless communication is efficient in air and has become a technology of choice for many wearable wireless sensors.

However, EM-based wireless communication is less efficient with implanted sensors, especially when sensors must be implanted inside the body, for example to collect data from inner organs.

An issue is that electromagnetic waves are attenuated by biological tissues and thus require high power to be adequately transmitted. However, using high power can damage the surrounding biological tissue and can be unsafe for the patient. Another issue is that the dimension of the RF antenna is difficult to shrink down in size because the attenuation of EM signals scales with the frequency of the EM signals. To get around this issue, instead of using an implanted RF antenna, some EM-based implantable sensors use wires to deport the RF antenna towards the skin surface or below the skin. However, the wires can generate discomfort and can trigger and propagate infections in the biological tissue.

There is therefore a need for to wireless and battery free sensors for biomedical applications capable of alleviating or overcoming at least part of the above drawbacks. It is particularly desirable to reduce the size of such wireless and battery free sensors in order to develop minimally invasive implantable technology.

### SUMMARY

An object of the present invention is to provide a sensor for implantation in an acoustically propagative medium such as biological tissue, comprising:
a solution-gated field effect transistor comprising an electrically conductive channel between a drain electrode and a source electrode of the transistor, the channel being able to be in contact with at least a portion of the surrounding medium,
an ultrasonic piezoelectric transducer element,
wherein the piezoelectric transducer element is electrically connected between the drain electrode and the source electrode of the transistor,
wherein the transistor is configured to act as a sensor for detecting a physiological parameter of interest surrounding media and to modulate the acoustic reflection coefficient of the piezoelectric transducer element depending on said parameter of interest.

In some optional embodiments, the sensor comprises a substrate, wherein the solution-gated field effect transistor and the piezoelectric transducer are attached to the substrate.

In some optional embodiments, the substrate is a flexible substrate.

In some optional embodiments, the substrate is a polymer substrate.

In some optional embodiments, the channel of the solution-gated field effect transistor is made of graphene.

In some optional embodiments, the channel of the solution-gated field effect transistor is deposited on a transistor support layer, wherein the transistor support layer is preferably made of a polymer such as parylene.

In some optional embodiments, the solution-gated field effect transistor and the piezoelectric transducer element are encapsulated in a biocompatible enclosure.

In some optional embodiments, the piezoelectric transducer element is a vibrating piezoelectric membrane.

In some optional embodiments, the piezoelectric transducer element is a bulk piezoelectric actuator.

In some optional embodiments, the sensor is configured to detect the presence or measure the concentration of a chemical element or compound in the medium.

In some optional embodiments, the sensor is a biosensor capable of detecting the presence of biological agents such as antigens in the medium.

In some optional embodiments, the channel of the transistor is functionalized with biological receptors.

In some optional embodiments, the sensor is configured to monitor a physiological signal, such as a neural signal, the parameter of interest to be measured being representative of said physiological signal.

According to another aspect of the invention, a measurement system comprises a sensor as described above, an external ultrasound measurement device comprising an ultrasound transducer, and a control and measurement interface connected to the external ultrasound measurement device, wherein the ultrasonic piezoelectric transducer is able to communicate wirelessly with the external ultrasound measurement device through the surrounding medium.

In some optional embodiments, the measurement system further comprises an electronic controller connected to the control and measurement interface for generating an excitation ultrasound wave and for measuring and analyzing an echo ultrasound wave reflected by the ultrasonic piezoelectric transducer.

### BRIEF DESCRIPTION OF THE DRAWINGS

The invention will be understood upon reading the following description, provided solely as an example, and made in reference to the appended drawings, which may not necessarily be to scale and in which certain features of the disclosure may be shown in somewhat schematic form:

FIG. 1 is a simplified block diagram of a measurement system comprising a biological sensor,

FIG. 2 is a simplified diagram of the sensor of FIG. 1,

FIG. 3 is a simplified illustration of the sensor of FIGS. 1 and 2 shown in an exploded view,

FIG. 4 illustrates examples of experimental results showing the transfer curve of ultrasonic echo vs. gate voltage of the sensor of FIGS. 1 and 2, measured with the measurement system of FIG. 1,

FIG. 5 is a block diagram of an exemplary method of operation of the sensor of FIG. 3.

### DETAILED DESCRIPTION OF SOME EMBODIMENTS

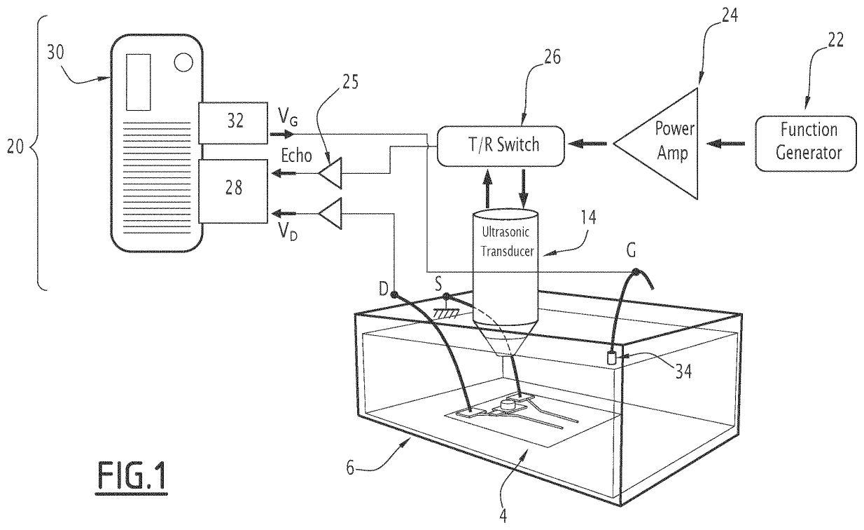

On FIG. 1 there is illustrated a measurement system 2 comprising a sensor 4.

According to embodiments of the invention, the sensor 4 is a wireless and battery free sensor for biomedical applications. The sensor 4 is preferably adapted to detect a physiological signal or a biological agent or a chemical agent in the medium 6, as will be made apparent in the following description.

It should however be noted that the sensor 4 can be used independently from the measurement system 2. Different embodiments of the measurement system 2 could be used with the sensor 4.

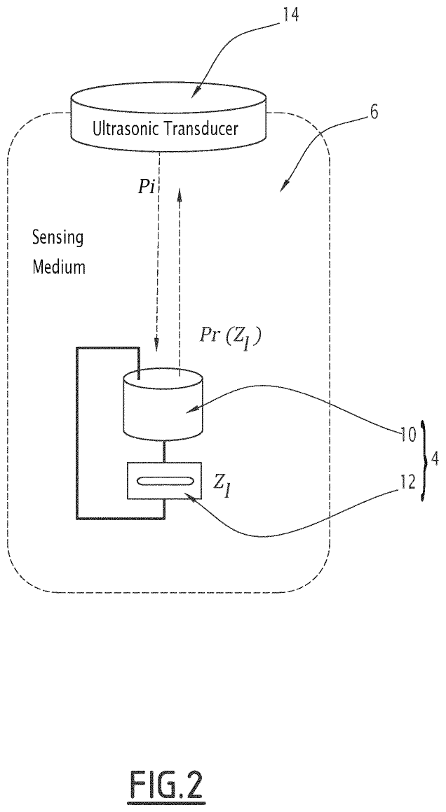

In the illustrated example, the sensor 4 is implanted or immerged in an acoustically propagative medium 6 (noted “sensing medium” on FIG. 2).

For example, the medium 6 is a biological tissue.

Other embodiments are possible, in which the medium 6 is a gel, or a fluid, or an electrolyte solution, or a soft material, for example. In some examples, animal meat can be used to simulate a biological tissue or an organ for testing purposes.

In this example, the medium 6 is a volume of saline solution (e.g., a phosphate buffered saline solution), used for testing purposes. In a non-limiting example, the saline solution is held in a tank having a rectangular shape.

In this description, the sensor 4 will be said to be “implanted” in the medium 6 when the sensor 4 is immersed in a fluid 6, as in the example of FIG. 1.

In preferred embodiments, the sensor 4 is an implantable biosensor.

The sensor 4 is preferably adapted to be implanted in the body of a living subject, such as an animal or a human. In such cases, the propagation medium 6 may comprise biological tissue such as, in a non-limiting list, flesh, bone, skin, nerves or brain matter, vascular elements such as veins or arteries, or one or more biological organs. The sensor 4 may be implanted at various depths the body, or implanted at an external surface of the body (e.g., as a skin patch).

However, the use of the system 2 and/or of the sensor 4 is not limited to medical or biomedical applications. For example, in some embodiments, the sensor 4 could be used for non-destructive testing applications, e.g. for real time monitoring of properties (such as mechanical properties) of a material.

As visible in FIG. 2, the sensor 4 comprises a wireless communication device 10 and a sensing device 12, or sensing element, connected to the communication device 10.

In preferred embodiments, the communication device 10 and the sensing device 12 are encapsulated in a housing to form an implantable device.

The wireless communication device 10 is an ultrasound capable communications device. In what follows, the wireless communication device 10 may be referred to as an ultrasound communication device 10.

For example, the wireless communication device 10 is a piezoelectric transducer element or actuator. In preferred embodiments, the piezoelectric transducer element is a vibrating piezoelectric membrane, although other embodiments are possible, such as a bulk piezoelectric actuator.

For example, in the illustrated embodiment, the wireless communication device 10 is a disc shaped bulk piezoelectric actuator.

The wireless communication device 10 is able to communicate wirelessly with an external communication device 14, such as an ultrasonic transducer placed at a distance from the communication device 10, using ultrasound waves propagating in the medium 6.

For example, the external communication device 14 may be placed at an external boundary of the medium 6. When the sensor 4 is implanted in a body (such as an animal body or a patient body), the external communication device 14 may be placed outside of the body albeit in direct contact with the body.

The wireless communication device 10 is capable of receiving one or more incident ultrasound waves (noted Pi on FIG. 2), e.g. an ultrasound waves emitted by the external communication device 14.

The wireless communication device 10 is further configured to emit (e.g. reflect) one or more reflected ultrasound waves (noted Pᵣ(Zₗ) on FIG. 2).

The sensing device 12 is configured to measure a parameter of interest in the medium 6, such as a physiological parameter of interest.

For example, the sensing device 12 is configured to act as a variable shunt load (a variable impedance) that changes its value depending on the parameter of interest. The impedance value (shunt load) of the sensing device 12 is noted Zₗ.

The ultrasound communication device 10 is configured to modulate an ultrasound echo wave, the modulated amplitude being a function of the state of the sensing device 12 (e.g., the value of the variable impedance of the sensing device 12, itself depending on the value of the parameter of interest measured by the sensing device 12).

More precisely, the sensing device 12 is able to modulate the acoustic reflective coefficient of the piezoelectric ultrasound communication device 10.

For example, the amplitude of the reflected ultrasound waves Pᵣ(Zₗ) is a function of the amplitude of the incident ultrasound waves Pᵢ and depends on the impedance value Zₗ (shunt load) of the sensing device 12.

In other words, the ultrasonic echo can be used to interrogate wirelessly the state of the transistor 12, for example using a pulsed-echo measurement technique with ultrasound waves through the medium 6.

The parameter of interest depends on the intended use of the sensor 4.

In some embodiments, the sensor 4 may be configured to detect or measure the concentration of a chemical element or compound in the medium 6. Thus, the parameter of interest to be measured is a concentration of a chemical element or of a chemical compound in the medium 6.

In some embodiments, the sensor 4 may be configured for biosensing applications, e.g. to detect the presence of biological agents such as antigens in the medium 6. Thus, the parameter of interest to be measured is a concentration of a biological substance or entity, such as an antibody.

In that case, a sensing portion of the sensing device 12 may be functionalized with appropriate biological receptors.

In some embodiments, the sensor 4 may be configured to monitor and/or record a physiological signal, such as a neural signal (e.g. an electrical or electrochemical signal generated by one or more neurons). Thus, the parameter of interest to be measured is representative of said physiological signal.

Many other applications are possible.

In preferred embodiments, the sensing device 12 is a solution-gated field-effect transistor (SG-FET). In what follows, the sensing device 12 may be referred to as the transistor SG-FET 12.

The SG-FET transistor 12 comprises a drain electrode, a source electrode and an electrically conducting channel extending between the drain and the source. The channel, or at least a portion of the channel, is in direct contact with the surrounding medium 6.

In many embodiments, the transistor 12 is configured to act as a sensor for detecting a physiological signal or a biological agent in the surrounding medium 6.

In some applications, the transistor 12 may be associated with a gate electrode (noted 34 on FIG. 1). The gate electrode 34 is configured to be in direct contact with the medium 6 so that during operation, the gate electrode is in electrical contact with the channel through the medium 6. In the example illustrated on FIG. 1, the gate electrode 34 is immersed in the saline solution 6. For example, the gate electrode 34 may be an Ag/AgCl gate electrode, other embodiments being nonetheless possible.

The gate electrode 34 may be advantageous in applications such as monitoring and/or recording a physiological signal, for example a neural signal. The gate electrode 34 can also be used for testing purposes, as in the illustrated example of FIG. 1.

However, in many applications and embodiments, the gate electrode 34 is not necessary and can be removed when the sensor 4 is implanted and in operation in the medium 6.

The ultrasound communication device 10 is electrically connected to the SG-FET transistor 12. More precisely, the piezoelectric transducer element 10 is electrically connected between the drain electrode and the source electrode of the transistor 12.

In further preferred embodiments, the channel of the SG-FET transistor 12 is made of graphene, although other materials can be used in alternative embodiments, such as electrically conductive inorganic materials, organic materials or carbon based materials.

For example, in alternative embodiments, the channel of the SG-FET transistor 12 may comprise an electrically conductive polymer such as PEDOT: PSS (poly(3,4-ethylenedioxythiophene)polystyrene sulfonate), or artificial diamond such as hydrogen-terminated diamond, among other examples.

Preferably, the transistor 12 is built on a flexible substrate, such as a polymer substrate, for example a polyethylene terephthalate (PET) substrate.

In other embodiments, the transistor 12 is built on a rigid substrate, such as a ceramic substrate or a diamond substrate.

Preferably, the ultrasound communication device 10 and the transistor 12 are integrated in a common device.

In many embodiments, the ultrasound communication device 10 and the transistor 12 are built on (or attached to) a common substrate.

For example, the ultrasound communication device 10 and the transistor 12 are encapsulated in a biocompatible enclosure, e.g. an enclosure made in a biocompatible material.

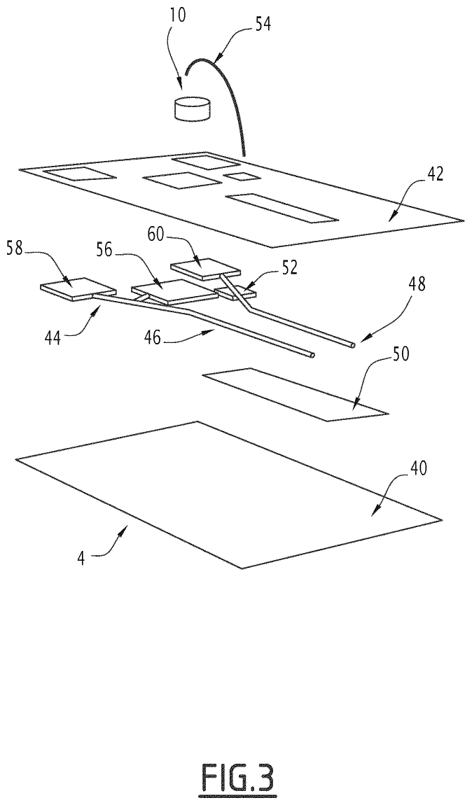

FIG. 3 illustrates a possible embodiment of the sensor 4 shown in an exploded view. This example is given for illustrative purposes. Different sensors designs and constructions details may be used in alternative embodiments.

The sensor 4 comprises a bottom substrate layer 40, a top substrate layer 42 and electrical contacts 44 sandwiched between the bottom substrate layer 40 and the top substrate layer 42.

It is to be understood that, in a final operational configuration of the sensor 4, the bottom substrate layer 40 and the top substrate layer 42 are superimposed onto each other.

The bottom substrate layer 40 and the top substrate layer 42 are preferably made of a flexible material, such as a polymer material, for example as described above.

For example, the bottom substrate layer 40 and the top substrate layer 42 are flat sheets having similar or identical dimensions.

The electrical contacts 44 comprise the drain electrode 46 and the source electrode 48, which in this example have an elongated strip shape. The drain electrode 46 and the source electrode 48 may be parallel to each other and have similar or identical lengths.

Preferably, the electrical contacts 44 are in metal, such as silver. Other suitable materials such as metals, or alloys, or electrically conductive materials (such as doped semiconductors) or combinations thereof can be used in alternative embodiments.

The sensor 4 comprises a channel layer 50, placed between and/or below the drain electrode 46 and the source electrode 48.

In preferred embodiments, the channel layer 50 may comprise a graphene layer deposited onto a transistor support layer, preferably a transistor support layer made of a polymer, such as a sheet of parylene or any suitable material. The transistor support layer may be in direct contact with an upper face of the bottom layer substrate 40.

The contacts 44 may also comprise contact pads at least for connecting the ultrasound communication device 10 with the source and the drain of the transistor 12.

For example, the contacts 44 comprise a first contact pad 52 connected to the source electrode 48 for connecting an upper terminal of the ultrasound communication device 10 with a suitable electrical connection element, such as a bonding wire 54.

The contacts 44 comprise a second contact pad 56 connected to the drain electrode 46 for connecting a lower terminal of the ultrasound communication device 10, e.g. by direct electrical contact.

The contacts 44 may comprise a third contact pad 58 and a fourth contact pad 60, respectively connected to the drain electrode 46 and the source electrode 48. The contact pads 58 and 60 may be used to electrically connect the source and the drain of the transistor to an external measurement system for testing purposes, using wires, as in the example of FIG. 1.

However, in normal use, the source and the drain of the transistor 12 are not necessarily connected by wires to a measurement device. Such direct connections are shown here only for explanatory purposes and may be omitted. The third contact pad 58 and the fourth contact pad 60 may be omitted from the sensor 4.

Preferably, the top substrate layer 42 comprises holes or openings formed over the contact pads for allowing an electrical contact with some or all of the contact pads.

The top substrate layer 42 further comprises one or more holes or openings aligned with the channel of the transistor 12 for allowing an electrical contact between the channel and the surrounding media 6.

Thus, in this example, the sensor 4 has a multilayer structure.

In preferred embodiments, the thickness of the sensor 4 is lower than or equal to 150 μm, preferably lower than or equal to 100 μm.

Other embodiments are possible.

An exemplary method of construction of the sensor 4 is now described for illustrative purposes.

First, the top and bottom substrate layers 40 and 42 are acquired. At least one of the aforementioned holes or openings are formed in the top substrate layer 42 for example by laser etching, at least to create an opening for the channel layer.

Then, electric contacts 44 are patterned on a first side of the top substrate layer 42, for example using a conductive ink printer.

The channel layer 50 is attached to the first face of the top substrate layer 42. For example, a graphene sheet deposited on a transistor support layer is cut to the appropriate size and placed in front of the opening formed on the top substrate layer 42.

The bottom channel layer 40 is glued the top substrate layer 42 to encapsulate the contacts 44. The holes or openings associated to the contact pads 52, 56, 58 and 60 are then created using the laser etching machine. The ultrasound communication device 10 and the wires (e.g. the bonding wire 54) are soldered using a silver paste. The device is finally sealed and encapsulated in an epoxy layer.

Other manufacturing methods can be used, especially if different materials are used for the components of the sensor 4.

Turning back to FIG. 1, there is illustrated a control and measurement interface 20 connected to an electronic controller 30 for driving and interacting with the sensor 4 when the sensor is immersed or implanted in the medium 6.

The electronic controller 30 may include a processor and a computer memory storage in which are stored executable instructions, commands, and control algorithms, as well as other data and information required to operate as described.

The computer memory may be, for example, a random access memory (RAM), and/or other forms of memory used in conjunction with such memory, such as flash memory (FLASH), and/or magnetic memory, and/or programmable read-only memory (PROM), and/or electronically erasable programmable read-only memory (EEPROM), and the like.

In this description, the expression “processor” may refer to electronic devices such as a microprocessor or a microcontroller, or to equivalent elements such as programmable logic controllers (PLC), application-specific integrated (ASIC) circuits, field-programmable gate array (FGPA) circuits, digital signal processing (DSP) circuits, graphical processing units (GPU), logic circuits, analog circuitry, equivalents thereof, and any other circuit or processor capable of executing the functions described herein.

In many embodiments, the electronic controller 30 is a computer, such as a personal computer, or a computer workstation, or a laptop, or equivalent.

The control and measurement interface 20 comprises an excitation portion and a measurement portion.

The excitation portion is connected to the external ultrasound communication device 14 and is configured to drive the external ultrasound communication device 14 with one or more excitation signals (such as pulses or waveforms) to emit one or more ultrasound waves in the medium 6, preferably towards the sensor 4.

For example, the control and measurement interface 20 comprises an electronic function generator 22 for generating one or more excitation signals, a power amplifier 24 connected to an output of the function generator 22, and a switch 26 connecting the output of the power amplifier 24 to the external ultrasound communication device 14. The external ultrasound communication device 14 is able to convert the received excitation signals into one or more ultrasound waves in the medium 6.

For example, the switch 26 is connected to input/output terminals of a driver circuit of the piezoelectric actuator of the device 14.

The excitation portion is connected to the external ultrasound communication device 14 and to the electronic controller 30.

The excitation portion is configured to measure the ultrasound waves reflected by the ultrasound communication device 10 of the sensor 4, using the external ultrasound device 14, for example using a pulse-echo measurement method. The ultrasound echo waves reflected by the device 10 and measured by the external ultrasound device 14 are converted into suitable signals (e.g., electrical signals) to be processed by the electronic controller 30.

In some embodiments, the control and measurement interface 20 comprises a signal acquisition interface 28 connected to the electronic controller 30 for acquiring signals measured by the external ultrasound device 14. The acquisition interface 28 may comprise one or more analog to digital converters.

For example, the switch 26 is connected signal acquisition interface 28.

An additional amplifier 25 may be used to amplify the signal collected at the output of the switch 26.

The switch 26 can switch automatically and reversibly between a first configuration state, in which the excitation portion of the control and measurement interface 20 is connected to the external ultrasound device 14, and a second configuration state in which the measurement portion of the control and measurement interface 20 is connected to the external ultrasound device 14.

For example, the electronic controller 30 may be configured to automatically control the switch 26.

In the illustrated example, for testing purposes, the control and measurement interface 20 is also configured to apply an excitation voltage (V_{G}) between the gate and the source of the transistor 12 and to measure a voltage between the drain and the source of the transistor 12 (voltage V_{D}).

This experimental setup is used to measure the transfer function of the transistor 12.

For example, the gate excitation voltage VG is swept within the electrochemical potential window of the electrode gate 34 (e.g., between −0.5 V and +0.3 V for the Ag/AgCl electrode 34 used in the example), for example to simulate a change in the physical parameter of interest. The drain-source voltage V_{D} is measured to verify that the impedance of the channel of the transistor 12 is properly modulated.

In this embodiment, a source terminal S is connected to the source of the transistor 12, for example using a wire or any suitable electric conductor connected to the contact pad 60 previously described. For example, the source terminal S is connected to an electrical ground.

A gate terminal G is connected to the gate electrode 34 using a wire or any suitable electric conductor. The gate terminal G is connected to a signal output interface 32 of the electronic controller 30. For example, the signal output interface 32 comprises a digital to analog converter.

A drain terminal D is connected to another input port of the acquisition interface 28 to the drain of the transistor 12, for example using a wire or any suitable electric conductor connected to the contact pad 58 previously described. An additional amplifier 25 may be used to amplify the signal collected from the drain terminal D.

Different embodiments of the measurement system 2 could be used with the sensor 4. The interface 20 could be built differently when it is not used for testing purposes. For example, in normal operation, when the sensor 4 is implanted in a target host, there is no need for a wired connection between the acquisition interface 28 and the source or drain of the transistor 12. Nor is there any need for the probe 34.

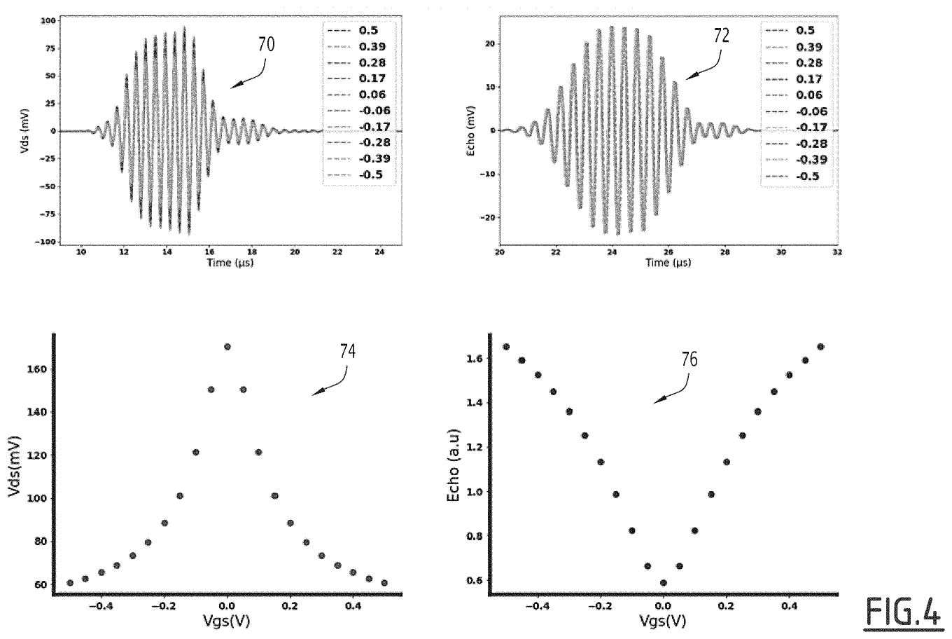

FIG. 4 illustrates an example of experimental results showing the transfer curve of ultrasonic echo vs. gate voltage of the sensor 4.

For example, the external ultrasonic transducer 14 is positioned on top of the sensor 4 under test to perform the pulse-echo measurement method.

During a transmission phase (pulse phase) the transducer 14 is excited by the wave function generator 22 to emit an ultrasonic wave. During a subsequent receiving phase (echo phase) the transducer 14 is connected through the switch 26 to the acquisition interface 28 to record the echo signal. The setup allows to measure the echo signal and the drain-source voltage across the drain and the source of the SG-FET transistor 12.

The first graph 70 of FIG. 4 illustrates the drain-source voltage (Vds, as y-axis, in millivolts) as a function of time (x-axis, in microseconds) for different values of the gate voltage V_{G} chosen in the aforementioned potential window (top right insert of the graph 70).

For each chosen gate voltage V_{G} value, one pulse ultrasound is emitted and the maximum amplitude of the echo is collected by the electronic controller 30.

The second graph 72 of FIG. 4 illustrates the amplitude of the measured ultrasound echo (Echo, as y-axis, in millivolts) measured by the external ultrasound transducer 14 as a function of time (x-axis, in microseconds) for the same different values of the gate voltage VG as described above (top right insert of the graph 72).

The third graph 74 of FIG. 4 illustrates the drain-source voltage (noted Vds, as y-axis, in millivolts) as a function of the gate voltage V_{G} (also noted Vgs) chosen in the aforementioned potential window (x-axis, in volts).

The fourth graph 76 of FIG. 4 illustrates the amplitude of the measured ultrasound echo (Echo, as y-axis, in arbitrary units) as a function of the gate voltage V_{G} (also noted Vgs) chosen in the aforementioned potential window (x-axis, in volts).

These results show that an ultrasound echo signal can be modulated by the gate voltage of the transistor 12 and that the modulated signal can be successfully transmitted through a propagation medium such a biological tissue.

Embodiments of the invention as described above are advantageous in many regards over the known art.

Most importantly, ultrasound wireless communications offer an advantageous alternative to the electromagnetic wireless communication technologies used in existing implantable biological sensors. One reason is that the attenuation of ultrasonic waves in biological tissue is weaker than the attenuation encountered with electromagnetic waves. Another reason is that the implantable piezoelectric transducer 10 required for wireless communication is much smaller than the radiofrequency RF antennas that would be required in comparable situations. Thus, wireless ultrasound communication allow a reduction the size of the sensor and make it possible to develop minimally invasive implantable sensors that can be implanted in a living body without causing unwanted side effects.

Additionally, solution-gated field-effect transistors require less space than transistors based on bulk semiconductor technologies such as CMOS (Complementary metal-oxide-semiconductor). Furthermore, solution-gated field-effect transistors transistors can be miniaturised more easily, and can be built easily on flexible substrates, making the sensor 4 even easier to implant in a body.

In addition, solution-gated field-effect transistors can be used for a wide range of healthcare applications such as biosensors or neural recording.

Thanks to its high electron mobility, graphene can be advantageously used as channel material in the transistors 12 especially for measurements in the high frequency regime (MHz or higher) which is a prime target for implantable biosensor technologies.

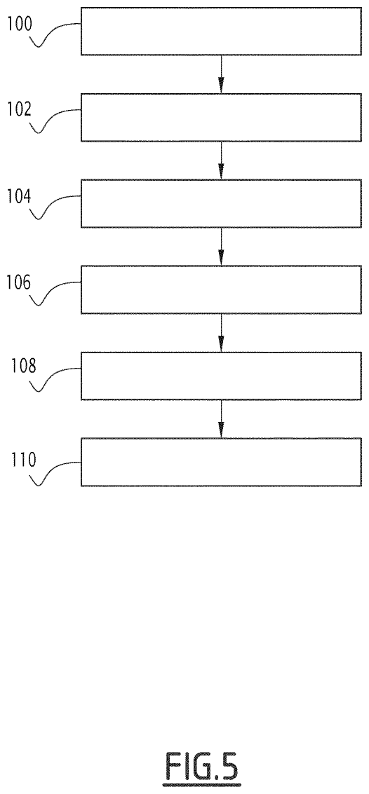

FIG. 5 illustrates an exemplary mode of operation of the sensor 4 given for non-limiting illustrative purposes.

The method starts at block 100.

At block 102, at least one excitation ultrasound wave is emitted by the external ultrasound communication device 14 in the medium 6 towards the sensor 4.

At block 104, the incoming ultrasound wave is reflected towards the external ultrasound communication device 14 by the ultrasound communication device 10 of the sensor 4. The acoustic reflection properties of the ultrasound communication device 10 depend on the resistance of the channel of the transistor 12, which depend on the value of the parameter of interest in the medium 6. In other words, the transistor 12 is coupled to the medium 6.

At block 106, the reflected ultrasound wave is measured by the external ultrasound communication device 14 after propagation through the medium 6.

At block 108, the electronic controller 30 receives and processes the measurement signals collected from the external ultrasound communication device 14.

For example, the electronic controller 30 may determine one of more value of the parameter of interest based on the echo signal measured by the external ultrasound communication device 14 in response to the excitation signal. For example, the electronic controller 30 may compare the value of the parameter of interest to a threshold value, and/or record different values over time of the parameter of interest, or identify a change over time of the value of the parameter of interest.

Optionally, at block 110, the electronic controller 30 may automatically determine a condition based on the determined values of the parameter of interest. For example, the electronic controller 30 may establish a diagnosis based on the identified values of the parameter of interest, or raise an alarm if the parameter of interest exceeds a predefined threshold.

In alternative embodiments, the method steps described above could be executed in a different order. One or more method steps could be omitted or replaced by equivalent steps. One or more method steps could be combined or dissociated into different steps.

The embodiments and alternatives described above may be combined with each other in order to create new embodiments of the invention within the scope of the claims.

1. A sensor for implantation in an acoustically propagative medium such as biological tissue, comprising:
a solution-gated field effect transistor comprising an electrically conductive channel between a drain electrode and a source electrode of the transistor, the channel being able to be in contact with at least a portion of the surrounding medium,
an ultrasonic piezoelectric transducer element,
wherein the piezoelectric transducer element is electrically connected between the drain electrode and the source electrode of the transistor,
wherein the transistor is configured to act as a sensor for detecting a physiological parameter of interest surrounding media and to modulate the acoustic reflection coefficient of the piezoelectric transducer element depending on said parameter of interest.

2. The sensor according to claim 1, further comprising a substrate, wherein the solution-gated field effect transistor and the piezoelectric transducer element are attached to the substrate.

3. The sensor according to claim 2, wherein the substrate is a flexible substrate.

4. The sensor according to claim 2, wherein the substrate is a polymer substrate.

5. The sensor according to claim 1, wherein the channel of the solution-gated field effect transistor is made of graphene.

6. The sensor according to claim 5, wherein the channel of the solution-gated field effect transistor is deposited on a transistor support layer, wherein the transistor support layer is preferably made of a polymer such as parylene.

7. The sensor according to claim 1, wherein the solution-gated field effect transistor and the piezoelectric transducer are encapsulated in a biocompatible enclosure.

8. The sensor according to claim 1, wherein the piezoelectric transducer element is a vibrating piezoelectric membrane.

9. The sensor according to claim 1, wherein the piezoelectric transducer element is a bulk piezoelectric actuator.

10. The sensor according to claim 1, wherein the sensor is configured to detect the presence or measure the concentration of a chemical element or compound in the medium.

11. The sensor according to claim 1, wherein the sensor is a biosensor capable of detecting the presence of biological agents such as antigens in the medium.

12. The sensor according to claim 11, wherein the channel of the transistor is functionalized with biological receptors.

13. The sensor according to claim 1, wherein the sensor is configured to monitor a physiological signal, such as a neural signal, the parameter of interest to be measured being representative of said physiological signal.

14. A measurement system, comprising a sensor according to claim 1, an external ultrasound measurement device comprising an ultrasound transducer, and a control and measurement interface connected to the external ultrasound measurement device, wherein the ultrasonic piezoelectric transducer element is able to communicate wirelessly with the external ultrasound measurement device through the surrounding medium.

15. The measurement system of claim 14, further comprising an electronic controller connected to the control and measurement interface for generating an excitation ultrasound wave and for measuring and analyzing an echo ultrasound wave reflected by the ultrasonic piezoelectric transducer element.
